(19) United States
(12) Patent Application Publication
(10) Pub. No.: US 2012/0276592 A1
(43) Pub. Date: Nov. 1, 2012

(54) Microfluidic System and Method for a Polymerase Chain Reaction

(75) Inventors: Martina Daub, Weissach (DE); Juergen Steigert, Stuttgart (DE); Christian Dorrer, Stuttgart (DE); Jochen Rupp, Stuttgart (DE)

(73) Assignee: Robert Bosch GmbH, Stuttgart (DE)

(21) Appl. No.: 13/452,855

(22) Filed: Apr. 21, 2012

(30) Foreign Application Priority Data
Apr. 27, 2011 (DE) 10 2011 017 596.2

(51) Int. Cl. C12P 19/34; C12M 1/00

(52) U.S. Cl. 435/91.2; 435/303.1

(57) ABSTRACT

A microfluidic system for a polymerase chain reaction is disclosed. The system includes a substrate having three chambers fluidically connected to one another in series, which chambers are held at different temperature levels. An elastic film on the substrate closes the chambers, wherein the chambers connected to one another in series are fluidically closable at the ends of the serial connection. The film above a chamber is movable into the chamber for emptying of the chamber. Thus, without a separate pump, it is possible for a PCR solution to be pumped through the chambers or temperature levels, with the PCR solution in a chamber acquiring the temperature thereof very rapidly.

This application claims priority under 35 U.S.C. §119 to patent application no. DE 10 2011 017 596.2, filed on Apr. 27, 2011 in Germany, the disclosure of which is incorporated herein by reference in its entirety.

The present disclosure relates to a microfluidic system for a polymerase chain reaction (PCR) and to a method for carrying out a polymerase chain reaction.

### BACKGROUND

In molecular diagnostics, the polymerase chain reaction (PCR) is often carried out in order to multiply DNA strands. In PCR, a PCR master mix containing the substances necessary for carrying out the PCR is added to the DNA. DNA and PCR master mix form the PCR solution. The PCR solution is repeatedly brought to three defined temperature levels, one after another. For this purpose, the standard approach is to use what are known as thermal cyclers. Systems in which the PCR takes place in a microfluidic system are known from the literature. WO 2001/007159 A2 describes a microfluidic device in which a single reservoir is brought successively to the different temperature levels.

Yao et al., Biomedical Microdevices 2005, 7, 253, use a long microfluidic channel in a microfluidic system. When DNA solution flows just once through the channel, it is conducted repeatedly across the different temperature zones. Here, an external pump is used.

A circular channel having three temperature zones is used by Chung et al., in IEEE MEMS 2011, 865, Cancun, MEXICO, 23-27 Jan. 2011, in a microfluidic system. No pump is used; instead buoyant forces are utilized.

### SUMMARY

The disclosure provides a microfluidic system as set forth below.

According to the disclosure, three microfluidic process chambers are provided, each of which is at a particular temperature level necessary for the respective PCR step. As a result of pumping into the process chamber exhibiting the respective temperature level, the PCR solution is brought to the temperature level. The PCR solution contains the DNA and a PCR master mix, with the PCR master mix containing the substances necessary for carrying out the PCR. The PCR solution is pumped between the process chambers by means of a film above the chambers, which is deflected into a chamber in a controlled manner in each case and alters the chamber volume.

The disclosure likewise provides a corresponding method as set forth below.

Further advantageous embodiments of the disclosure will be apparent from the description below.

According to the disclosure, the microfluidic chambers are at a constant temperature level. Only the liquid is heated up or cooled down. As a result, the thermal mass of the system is greatly reduced and the PCR can take place very much faster than in systems using thermal cyclers.

In the case of conventional instruments, considerable effort is expended in order to achieve rapid cooling, for example by means of cooling using Peltier elements. In contrast, an instrument for thermal control of the present disclosure can be constructed in a distinctly simpler and more economical manner, for example when resistance heating elements are used.

As a result of using the film above the process chambers for pumping, there is no need for an additional pump, the space requirement is lower and the liquid cannot evaporate.

### BRIEF DESCRIPTION OF THE DRAWINGS

FIG. 1 shows a perspective view of a section from a microfluidic system according to one embodiment of the present disclosure having external pump activation.

FIG. 2 shows diagrammatically a substrate layer comprising fluidic elements from FIG. 1 in several activation states A to D.

FIG. 3 shows an exploded perspective view of a section from a microfluidic system according to a further, integrated embodiment of the present disclosure having internal pump activation.

FIG. 4 shows diagrammatically a process chamber arrangement of a microfluidic system according to a further embodiment of the present disclosure with cyclic filling of process chambers.

FIG. 5 shows diagrammatically a process chamber arrangement of a microfluidic system according to another embodiment of the present disclosure with filling of process chambers by means of back-and-forth pumping.

FIG. 6 shows a flow chart of the method for carrying out a polymerase chain reaction in a microfluidic system according to one embodiment of the present disclosure.

### DETAILED DESCRIPTION

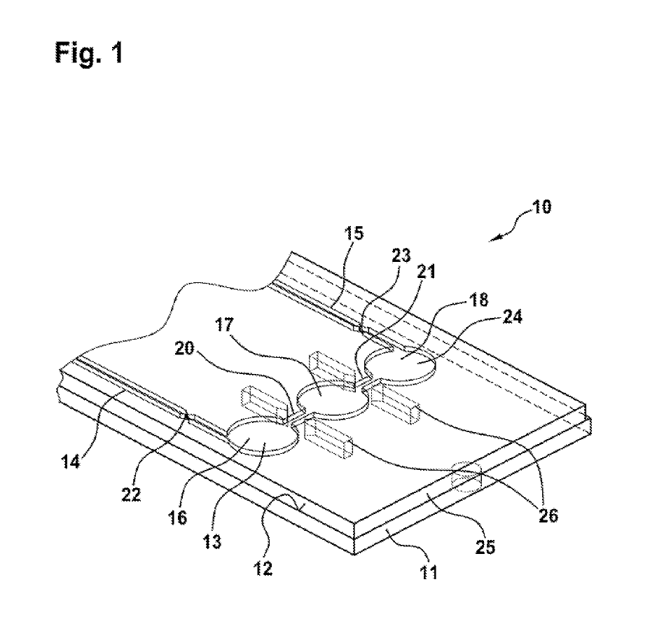

FIG. 1 shows a perspective view of a microfluidic system 10 according to one embodiment of the present disclosure. A substrate 11, in this case a polymer substrate, contains on an upper side 12 a fluidic structure 13 having an inlet channel 14, an outlet channel 15 and three chambers fluidically connected to one another in series, viz. a first chamber 16, a second chamber 17 and a third chamber 18. The first chamber 16 is connected to the second chamber 17 via a short connecting channel 20, and the second chamber 17 is connected to the third chamber 18 via a short connecting channel 21. The inlet channel 14 has an inlet valve 22, and the outlet channel 15 has an outlet valve 23. The inlet valve 22 and the outlet valve 23 form controllable closures at the ends of a serial connection 24 comprising the serially connected chambers 16, 17 and 18.

The microfluidic system 10 has an elastic film 25, composed of a thermoplastic elastomer (TPE) for example, on the substrate 11. The film 25 is connected to the substrate 11 on the upper side 12 and closes the cavities of the fluidic structure 13. The elastic film 25 above the chambers 16, 17 and 18 is movable into the respective chamber for emptying of the chamber.

In this embodiment, control of the positions of the film segments above the chambers 16, 17 and 18 or deflection of the film segments into the chambers is effected externally by means of a stamp for each film segment. The stamp is driven, for example, by means of air pressure, but a thermomagnetic and/or magnetic drive is also possible. Similarly, the valves 22 and 23 are activated externally. In an alternative embodiment which is not shown, the valves are not integrated into the system, but are instead external components.

During operation, the chambers 16, 17 and 18 are at different temperature levels for the PCR and are held at these temperature levels. Here, the temperature is controlled externally from below, but can also be controlled internally, for example with heating elements, resistance elements, microwaves and/or by thermal radiation. In order to thermally insulate the chambers 16, 17 and 18 from one another, holes 26 are introduced into the substrate 11 next to the connecting channels 20 and 21, in each case on both sides.

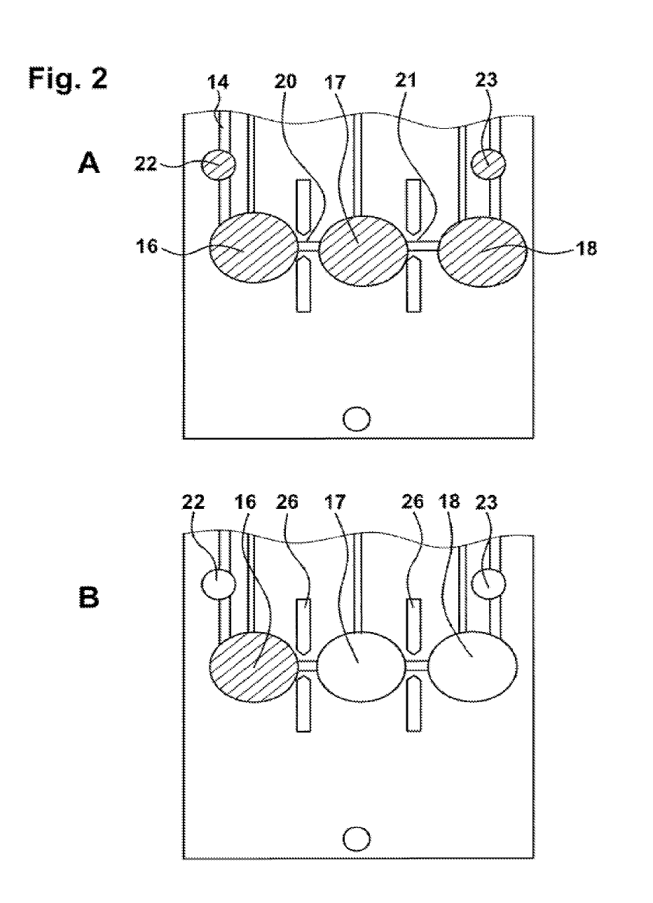

FIG. 2 illustrates the operation of the microfluidic system 10 in one embodiment, in which the PCR is carried out by pumping the PCR solution back and forth in the three chambers 16, 17 and 18 fluidically connected to one another in series. To this end, the substrate layer 11 comprising the fluidic elements which is known from FIG. 1 is shown in several activation states A to D. Each reaction chamber is in a temperature zone and is heated from below and/or above by an inherent heating element, for example a resistance heating element or a Peltier element. Adjacent chambers are connected via the connecting channels 20 and 21. The outer chambers 16, 18 are contacted by the inlet channel 14 and the outlet channel 15, through which the PCR solution can be flushed in and flushed out, respectively. The inlet and outlet channels can be closed by means of the valves 22 and 23. For better thermal insulation of the temperature zones against one another, the holes 26 are located between the chambers. The volume of the chambers 16, 17 and 18 is alterable by deflection of the elastic film 25 into the respective chamber. The state without deflection of the film into a chamber is referred to hereinafter as “chamber open”. The state with the furthermost deflection of the film into a chamber is referred to hereinafter as “chamber closed”. In this state, the liquid, i.e. in the case of PCR the PCR solution, is substantially displaced from the chamber volume, with at best residual liquid remaining.

The functional principle of the microfluidic system 10 is as follows:

Activation state A serves for the preparation of the PCR. The valves 22 and 23 and the chambers 16, 17, 18 are open. The PCR solution is flushed through the inlet channel 14 into the first chamber 16. The other two chambers 17 and 18 remain substantially empty. The temperature zones are brought to the target temperature. Exemplary values are: first chamber 95° C.; second chamber 72° C.; third chamber 55° C.

Then, the inlet valve 22, second chamber 17, third chamber 18 and the outlet valve 23 are closed in succession. In this activation state B, the PCR solution is now located substantially in the first chamber 16 and, after a short time, acquires the temperature prevailing there. The serial connection 24 of the serially connected chambers 16, 17 and 18 is substantially bubble-free.

The PCR solution is left in the first chamber 16 for a desired holding period, for example for 10 s. Denaturation of the DNA strands takes place.

Then, the second chamber 17 is opened, the first chamber 16 is closed and activation state C is reached. In this state, the PCR solution is now substantially in the second chamber 17.

Immediately thereafter, the third chamber 18 is opened and the second chamber 17 is closed and activation state D is reached. As a result of this sequence, the PCR solution is displaced into the third chamber 18 and, after a short time, acquires the temperature prevailing there. For example, a volume of about 1-10 μl of PCR solution acquires the temperature in the chamber within about 100 ms.

The PCR solution is left in the third chamber 18 for the desired holding period, for example for 10 s, and the DNA strands hybridize to primers.

Subsequently, the second chamber 17 is opened and the third chamber 18 is closed. As a result, the PCR solution is displaced into the second chamber 17 and activation state C is reached once more. After a short time, the PCR solution acquires the temperature prevailing there.

The PCR solution is left in the second chamber 17 for the desired holding period, for example for 10 s, and elongation takes place.

Finally, the first chamber 16 is opened and the second chamber 17 is closed. As a result, the PCR solution is displaced into the first chamber 16 and activation state B is reached once more. A complete PCR cycle has thus been described. This PCR cycle is then repeated according to the desired number of PCR cycles, for example 30 times.

After the desired number of PCR cycles has been reached, the valves 22 and 23 and the chambers 16, 17, 18 are opened and the PCR solution is flushed out.

The temperature zones can also be arranged in other orders. In this case, the control sequence changes accordingly. The above-described arrangement, however, has the advantage that the temperature gradients are minimized.

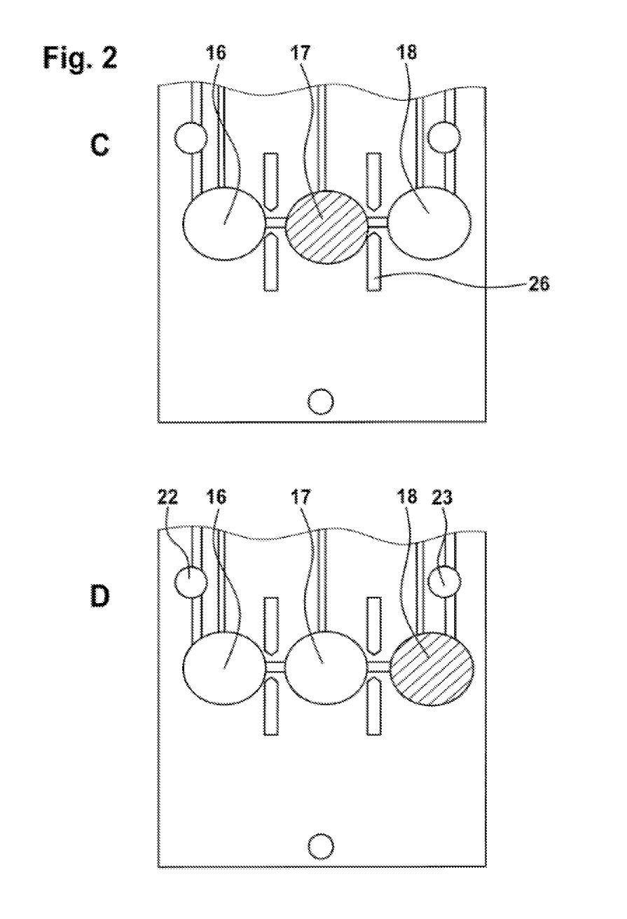

FIG. 3 shows an exploded perspective view of a section from a microfluidic system 30 according to a further, integrated embodiment of the present disclosure, in which pump activation is integrated. In order to show the layer structure, the individual layers are shown in exploded view. The microfluidic system 30 contains the elements known from FIG. 1 with the same reference numbers. These are the substrate 11 with the elements thereof and the elastic film 25. Arranged on the film 25 are a further substrate layer 31 comprising a pneumatic structure 32 and a cover layer 33. Activation of the valves and deflection of the film above the chambers is no longer effected externally in this embodiment, but internally by means of the elements of the pneumatic structure 32.

The further substrate layer 31 contains the pneumatic structure 32 punched out and aligned with respect to the fluidic structure 13 in substrate 11, with the activatable elements in substrate 11 having corresponding elements in the further substrate layer 31. The activatable elements in substrate 11 and the corresponding elements lie on top of one another with the elastic film 25 inbetween. Thus, pneumatic valve chambers 34, 35 correspond to the valves 22, 23, and pneumatic chambers 36, 37, 38 correspond to the chambers 16, 17, 18. Pneumatic action in these chambers 36, 37, 38 causes in each case deflection of the elastic film 25 into the activatable elements in substrate 11 and thus activation of these elements. Each of the pneumatic valve chambers 34, 35 is connected to an assigned pneumatic channel 40, 41, and each of the pneumatic chambers 36, 37, 38 is connected to an assigned pneumatic channel 42, 43, 44. The pneumatic valve chambers 34, 35 and pneumatic chambers 36, 37, 38 are pneumatically activated via these channels. The further substrate layer 31 additionally contains holes 45, which lie opposite the holes 26 and are separated therefrom by the film 25.

The cover layer 33 brings about pneumatic sealing of the pneumatic structure 32 and protects the microfluidic system 30 mechanically.

Instead of pneumatic operation of the pneumatic structure, it is also possible to carry out hydraulic operation with the same structure.

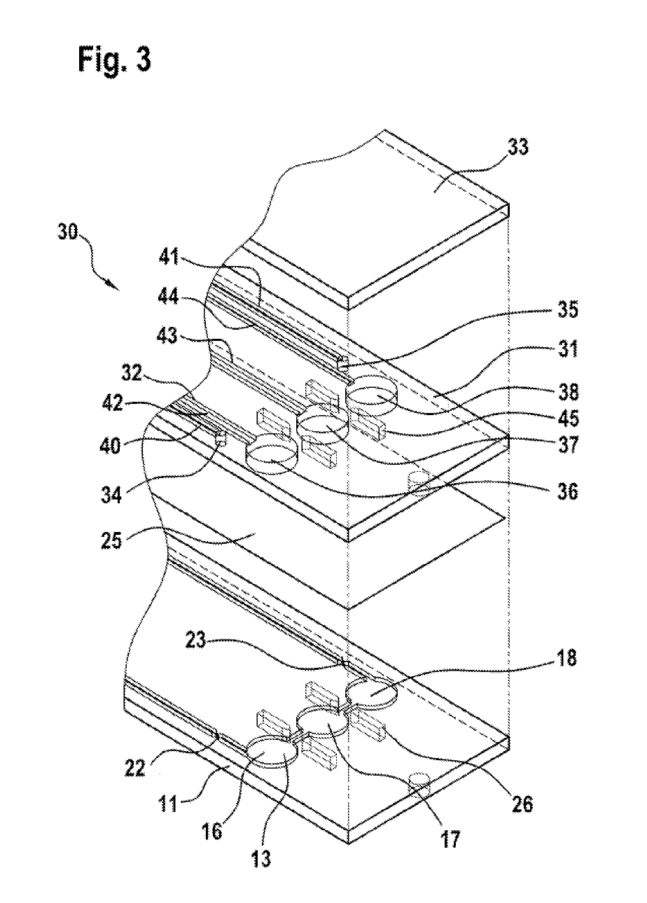

FIG. 4 shows diagrammatically a process chamber arrangement 50 of a microfluidic system according to a further embodiment of the present disclosure with filling of process chambers by means of back-and-forth pumping. The process chamber arrangement 50 corresponds to the arrangement of the chambers in FIG. 1 and to the operation of the chambers which is described in FIG. 2. Chambers 51, 52, 53 are fluidically connected in series, with an inlet channel 54 having an inlet valve 55 and an outlet channel 56 having an outlet valve 57. The chambers 51, 52, 53 are each at an assigned temperature level. Before and after PCR, when the valves 55, 57 are opened, the PCR solution is fed and conducted away via the channels 54, 56. During PCR, the PCR solution is pumped back and forth between the chambers 51, 52, 53 for PCR cycles. The flow direction of the PCR solution is indicated by arrows.

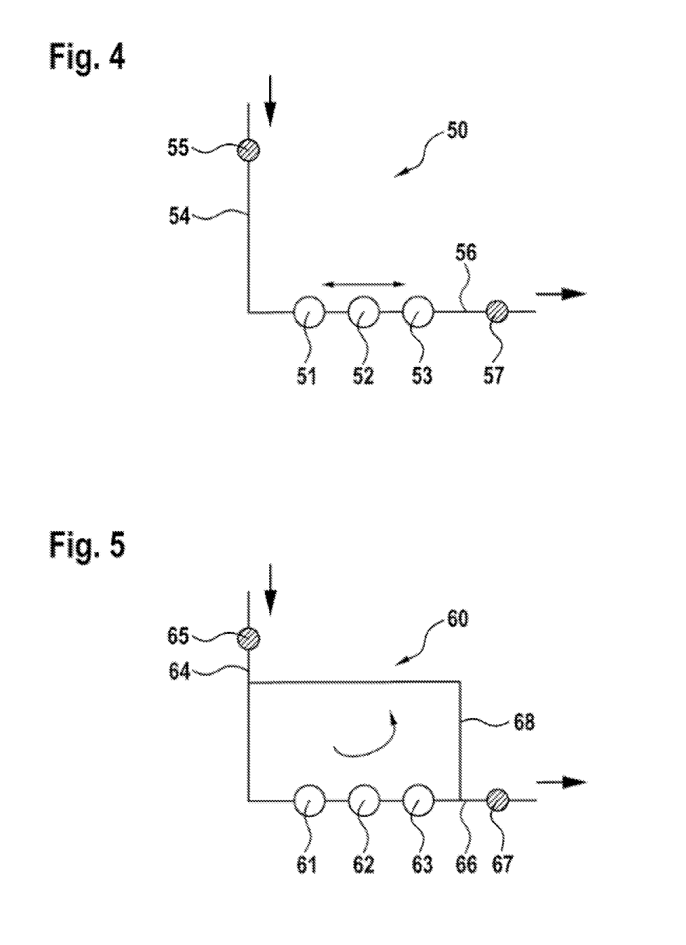

FIG. 5 shows diagrammatically a process chamber arrangement 60 of a microfluidic system according to another embodiment of the present disclosure with cyclic filling of process chambers. Chambers 61, 62, 63 are fluidically connected in series, with an inlet channel 64 having an inlet valve 65 and an outlet channel 66 having an outlet valve 67. In addition, process chamber arrangement 60 has a connecting channel 68 which directly connects the outer chambers 61, 63 to one another. The chambers 61, 62, 63 are each at an assigned temperature level.

Before and after PCR, when the valves 65, 67 are opened, the PCR solution is fed and conducted away via the channels 64, 66. During PCR, the PCR solution is pumped cyclically between the chambers 61, 62, 63 for PCR cycles. The flow direction of the PCR solution is indicated by arrows. When assigning temperature levels to the chambers, the order in which the PCR solution flows through the chambers must be noted.

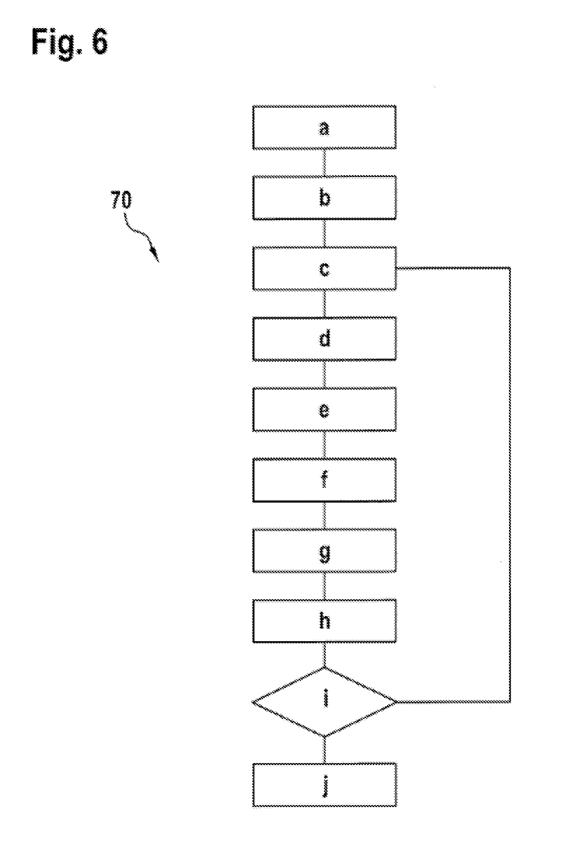

FIG. 6 shows a flow chart 70 of the method for carrying out a polymerase chain reaction in a microfluidic system according to one embodiment of the present disclosure. The microfluidic system has first, second and third chambers which are fluidically connected to one another in series, for example chambers 51, 52, 53 or the chambers 61, 62, 63. The method begins with method step a), adjusting the temperature of the chambers to a predefined temperature in each case. This is followed by method step b), selecting the number of PCR cycles. Method steps c) to h) are effected for this number of PCR cycles.

An individual PCR cycle begins with method step c), pumping a PCR solution into the first chamber. There, in method step d), the PCR solution is held for a first time interval. Subsequently, in method step e), the PCR solution is pumped into the second chamber. There, in method step f), the PCR solution is held for a second time interval. Then, in method step g), the PCR solution is pumped into the third chamber. There, in method step h), the PCR solution is held for a third time interval. The individual PCR cycle is now complete.

In method step i), the number of executed PCR cycles is compared with the number of predefined cycles and, if this is not yet reached, branched back to c). Otherwise, the PCR is ended and, in method step j), the PCR solution is pumped out.

Method steps a), b) and the first instance of carrying out method step c) can take place in any desired order. Pumping from a starting chamber into a target chamber is effected by means of controlled deflection of a film above the starting chamber into the starting chamber. The PCR solution is displaced from a filled open chamber by closure thereof and escapes into an adjacent empty open chamber, as described with reference to the microfluidic system 10 from FIG. 1 in conjunction with FIG. 2, where further details of the method are described. Owing to the closed valves 22, 23 and to a closed chamber not involved in the current pumping process in each case, the PCR solution cannot escape other than into the empty open chamber.

What is claimed is:

1. A microfluidic system for a polymerase chain reaction, comprising:
a substrate which includes three chambers for different temperature levels that are fluidically connected to one another in series,
an elastic film positioned on the substrate, said elastic film configured to close the chambers,
wherein the three chambers are connected to one another in series,
wherein the three chambers are fluidically closable at the ends of the serial connection, and
wherein, in each case, the film above a chamber is movable into the chamber for emptying of the chamber.

2. The microfluidic system according to claim 1, further comprising a drive configured to move the film above each of the three chambers.

3. The microfluidic system according to claim 2, further comprising, above each chamber, a pump chamber positioned adjacent to the film.

4. The microfluidic system according to claim 3, wherein the pump chambers are subjected to pneumatic action.

5. The microfluidic system according to claim 3, wherein the pump chambers are subjected to hydraulic action.

6. The microfluidic system according to claim 2, wherein the microfluidic system is configured for interaction with an external drive for moving the film above each chamber.

7. The microfluidic system according to claim 6, wherein the external drive comprises plungers.

8. The microfluidic system according to claim 1, further comprising valves at the ends of the serial connection.

9. The microfluidic system according to claim 1, further comprising a direct connecting channel between the outer chambers.

10. A method for carrying out a polymerase chain reaction in a microfluidic system having first, second and third chambers fluidically connected to one another in series, comprising:
a) adjusting the temperature of the chambers to a predefined temperature in each case;
b) selecting the number of PCR cycles;
for the number of PCR cycles
c) pumping a PCR solution into the first chamber;
d) holding the PCR solution in the first chamber for a first time interval;
e) pumping a PCR solution into the second chamber;
f) holding the PCR solution in the second chamber for a second time interval;
g) pumping a PCR solution into the third chamber;
h) holding the PCR solution in the third chamber for a third time interval;
wherein steps a), b) and the first instance of carrying out step c) can take place in any desired order,
wherein pumping from a starting chamber into a target chamber is effected by controlled deflection of a film above the starting chamber into the starting chamber.

11. The method according to claim 10, wherein a drive for the deflection of the film is in the microfluidic system.

12. The method according to claim 11, wherein the drive above each chamber comprises a pump chamber adjacent to the film.

13. The method according to claim 12, wherein the pump chambers are subjected to pneumatic action.

14. The method according to claim 12, wherein the pump chambers are subjected to hydraulic action.
